(19) United States
(12) Patent Application Publication
(10) Pub. No.: US 2003/0194817 A1
(43) Pub. Date: Oct. 16, 2003

(54) Device for use in detection of airborne contaminants

(76) Inventor: Kenneth P. Glynn, Raritan, NJ (US)

Correspondence Address: KENNETH P. GLYNN, ESQ., Glynn & Associates, P.C., 24 Mine Street, Flemington, NJ, 08822, US

(21) Appl. No.: 10/120,223

(22) Filed: Apr. 10, 2002

(51) Int. Cl. G01N 1/22; G01N 33/00

(52) U.S. Cl. 436/181; 436/183; 422/50; 422/61; 422/83; 422/88; 422/99

(57) ABSTRACT

A device for use in detection of airborne contaminants, and a method of using the device. The device includes: a flexible substrate having a front side and a back side; a contaminant carrier located on said front side; an adhesive located on said back side; and, a unique identifier located on one of said front side and said back side. The contaminant carrier may be a material adapted to receive and chemically bond with an airborne contaminant; adapted to receive and ionically bond with an airborne contaminant; or adapted to receive and physically hold an airborne contaminant.

### BACKGROUND OF THE INVENTION

1. Field of the Invention

The present invention relates to devices for use in detection of airborne contaminants, and more particularly to such devices which are used to detect exposure by a person to particular airborne contaminants. Soldiers and civilians may be exposed to undesirable contaminants which are airborne, such as mustard gas, poison gas, anthrax or other debilitating or fatal spores, etc. The present invention devices reduce uncertainty of exposure and include provision for tracking exposure issues by unique identifiers, which may be used to indicate times, places, persons or any combination thereof.

2. Information Disclosure Statement

The following patents are representative of the state of the art regarding toxin collection and identification as it pertains to the present invention:

U.S. Pat. No. 6,361,962 describes methods, materials, and systems for detecting toxins. In one aspect, a toxin contamination detector includes a substrate on which a bar code is printed. The bar code has a first color (e.g., black) that is effective to reflect light from a bar code scanning device to produce a bar code result. A toxin indicator is also included. The toxin indicator has a second color in the absence of toxin, which second color does not substantially affect or alter the bar code result. However, the toxin indicator presents a third color in the presence of toxin which substantially changes the bar code result; thereby indicating the presence of toxin.

U.S. Pat. No. 6,329,156 describes a protective antigen (PA) of Bacillus anthracis which is integral to the mechanism of anthrax poisoning. The cloning, expression and purification of a 32 kDa B. anthracis PA fragment (PA32) is described. This fragment has also been expressed as a fussion construct to stabilize green fluorescent protein (EGFP-PA32). Both proteins were capable of binding to specific cell surface receptors as determined by fluorescent microscopy and a flow cytometric assay. To confirm binding specificity in the flow cytometric assay, non-fluorescent PA83 or PA32 was used to competitively inhibit fluorescent EGFP-PA32 binding to cell receptors. This assay can be employed as a rapid screen for compounds which disrupts binding of PA cells. Additionally, the high intracellular expression levels and ease of purification make this recombinant protein an attractive vaccine candidate or therapeutic treatment for anthrax poisoning.

U.S. Pat. No. 6,317,080 describes a method of tracking airborne substances including the steps of detecting the presence of one or more airborne substances and releasing a tracking balloon into the path of the one or more airborne substances, the tracking balloon having a transmission means and a global positioning means adapted to communicate the latitude and longitude coordinates of the tracking balloon whereby the latitude and longitude coordinates of the tracking balloon are representative of the latitude and longitude of the one or more airborne substances previously detected.

U.S. Pat. No. 6,066,295 describes a method and device for remotely analyzing an agent dispersed in the atmosphere is disclosed in which a body is dispensed into a volumetric region of the atmosphere overlapping the region containing the agent. The body is tailored to induce changes in an electromagnetic response of the volumetric region including, the agent due to body the reactive probe and the agent. By sensing electromagnetic radiation from the region, information may be gained concerning the agent based upon the new electromagnetic response of the volumetric region induced by the reactions. Since a probe is used, source need not be known, and the electromagnetic signature may be tailored to atmospheric and device limitations.

U.S. Pat. No. 5,876,960 describes a method effective for the detection and quantification of bacterial spores in sample medium. A lanthanide such as europium or terbium is combined with a medium to be tested for endospore content. The lanthanide will react with calcium dipicolinate present in any bacterial spores in the sample medium to produce a lanthanide chelate, specifically, terbium or europium dipicolinate. The lanthanide chelate has distinctive absorbance and emission spectrums that can be detected using photoluminescence testing, for example. The occurrence of emission from the sample medium upon excitation at wavelengths, distinctive of the lanthanide chelate thus reveals the presence of spores in the sample medium. The concentration of spores can be determined by preparing a calibration curve that relates absorbance or emission intensities to spore concentrations for test samples with known spore concentrations. The calibration curve can be used to determine the spore concentration of a sample medium using the absorbance or emission intensity for the combined lanthanide-sample medium.

Notwithstanding the prior art, the present invention is neither taught nor rendered obvious thereby.

### SUMMARY OF THE INVENTION

The present invention is a device for use in detection of airborne contaminants, and, more specifically, it is a device to be affixed to a person so that when the person has been exposed to airborne contaminant(s), the airborne contaminants will be picked up by the device for subsequent analysis. Thus, the present invention device is a flexible unit with adhesive that is attached to the person for movement with the person through potential exposure to airborne contaminants. By “attached to the person” is meant physically adhered to the skin, or to the clothing of the person (anything worn by the person, including inner wear, outer wear, footwear and headwear), or to equipment on the person (a rifle, a backpack, a parachute or any other item that travels with the person). The device is flexible to conform to the contours of the surface to which it is attached, and may be tracked and/or stored, due to the unique identifier, which is an integral part thereof.

The present invention device includes:

(a.) a flexible substrate having a front side and a back side;

(b.) a contaminant carrier located on said front side;

(c.) an adhesive located on said back side;

(d.) a unique identifier located on at least one of said front side and said back side.

The contaminant carrier is a material adapted to receive and hold one or more airborne contaminants, and may take any form or combinations of forms that function to receive and contain the contaminant(s). For example, the contaminant carrier may recive and chemically bond with an airborne contaminant. Alternatively, it may receive and ionically bond with an airborne contaminant. Alternatively, it may receive and biologically bond with or react to an airborne contaminant. Or, it may simply receive and physically hold an airborne contaminant. It may include combinations of the foregoing.

Chemical bonding may occur when a reactant that is known to chemically combine with an airborne contaminant is included in the carrier. Likewise, ionic bonding occurs when a salt, an ionic polymer or other ionic material is included in the carrier, and the airborne contaminant is itself ionic and is attracted to oppositely charged ions in the carrier. Biological reaction occurs when a host organism is included in the carrier that will capture, consume, or combine in some way with the airborne contaminant, in this case a virus, spore, or other biological contaminant. As to those materials that physically hold a contaminant, these may be selected from adhesive materials, high surface area materials, and combinations thereof. The adhesives may be generic types, such as in common adhesive tape, or may be gels or jellies. The high surface area material may be microporous polymeric material or powdered inorganic material. Thus, these high surface area materials could be activated carbon, micro, or nano-type entrapments, such as honeycombs or they could be more sophisticated, such as molecular sieve type products. This physical capturing, thus, may involve sticking or trapping, or combinations thereof.

The unique identifier utilized in the present invention may be located directly on the flexible substrate or on its own flexible substrate attached to or affixed to the substrate of the device. For example, the unique identifier might be printed, perforated, or embossed onto the device flexible substrate, or on a separate sheet, which is attached to the device substrate. The unique identifier may involve numbers, letters, symbols, bars or any other indicia of information, and may represent names, dates, i.d. numbers, such as military serial numbers or social security numbers, or groupings of people or places, or any combination thereof. It may stand alone and be read directly and understood, or it may reference a data collection, as would an Army dog tag serial number, or it may need decoding or decryption. Thus, the unique identifier of the present invention may be selected form the group consisting of representations of alphabet, numerals, bar code, symbols and combinations thereof. The unique identifier may be located on the front side, on the back side, or on both sides of the flexible substrate. Alternatively, the unique identifier may be located in a first area of the front side, and the carrier located in a second area of the front side; or the identifier may be on the backside in an area different from the area containing adhesive, or under the adhesive.

The present invention also involves a method of creating and storing airborne contaminant samples and identification data for subsequent detection. This method includes:

(A.) applying a device for use in detection of airborne contaminants to a person, wherein the device is any embodiment of the above-described devices;

(B.) subjecting the person with the device to an airborne contaminant, wherein the airborne contaminant is received and held by said device; and,

(C.) subsequently subjecting the device with the airborne contaminant to a detection method, and correlating results from the detection method to the unique identifier.

In the present invention method, the device may be attached to the person by attaching the device to at least one element selected from the group consisting of skin of the person, clothing of the person, equipment of the person, and a secondary holder attachable to one of the foregoing.

### BRIEF DESCRIPTION OF THE DRAWINGS

The present invention should be more fully understood when the specification herein is taken in conjunction with the drawings appended hereto wherein:

FIG. 1 illustrates a front perspective view of a present invention device;

FIG. 2 shows a front perspective view of another present invention device utilizing a bar code identifier;

FIG. 3 shows yet another alternative embodiment present invention device;

FIGS. 4 and 5 show exaggerated side views of various present invention devices; and,

FIG. 6 shows a front view of another present invention device.

### DETAILED DESCRIPTION OF THE PRESENT INVENTION

The present invention is a device and a method used in detection of airborne contaminants. More specifically, it is a device for collecting airborne contaminants to which an individual has been exposed for subsequent detection or confirmation that the individual was or was not exposed to a selected airborne contaminant or contaminants. Thus, if a user wears a present invention device, the airborne contaminant is captured and is preserved for subsequent analysis. In some cases, analysis may be performed as a matter of course, e.g. mandatory; in other cases, analysis may be random; in yet other situations, the devices may be stored, and testing may be performed if a situation in hindsight becomes suspect. There are many important features of the present invention devices. They are adhesive and thus attachable to a person's skin, clothing, or equipment. They are flexible and will conform to the topographies to which they are attached. They capture the airborne contaminants for later analysis, as desired. They eliminate the need for persons to have blood tests or other body-present tests for exposure determination. They may be sealed and preserved for future analysis. They are trackable and create histories based on identifier parameter set by the administrator of their uses. They are useful in mobile environments, both those wherein a person moves through a potentially contaminated area, and wherein contaminated are moved past a person. The identifier may represent a group, e.g. an army battalion, an individual, a date, a time frame, a location, or any combination of these.

The identifier may be located on the front, the back, or both the front and the back of the device, and may be printed, embossed, hand written or otherwise applied, or a combination. For example, a soldier could be provided with a pack of devices all having that soldier's dog tag number. A spore could be provided wherein each day the soldier would write in the date before applying the device, e.g. to the forehead, chest, back of hand. Alternatively, the devices could include identifiers with a single dog tag number and a series of sequential numbers which would represent the first, second, third, etc. days on the battlefield. Many other alternative data combinations could be used in the identifier.

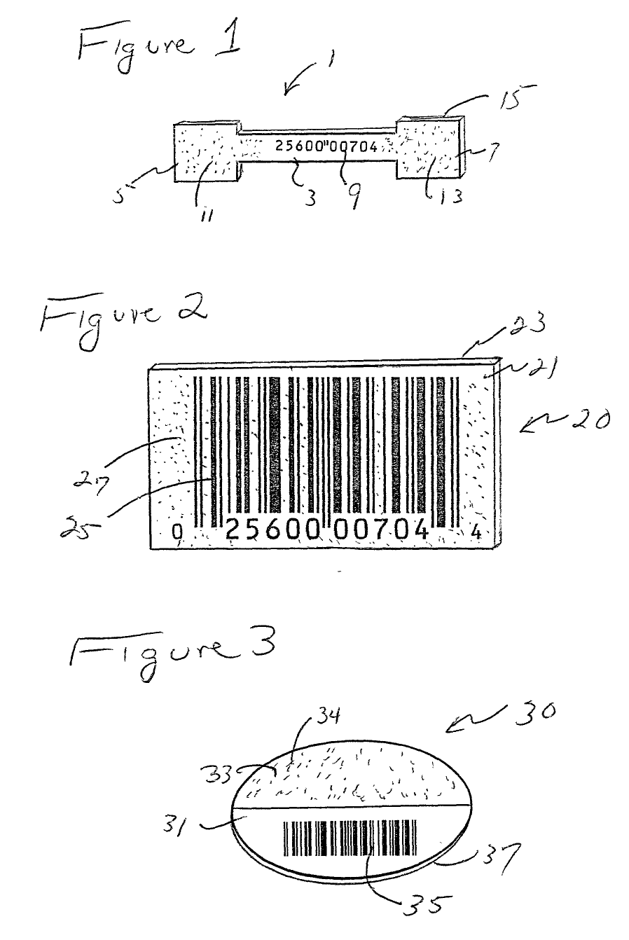

FIG. 1 shows one embodiment of a present invention device 1, having a front 3 and a back 15. Front 3 has a central portion with a unique numerical identifier 9, while ends 5 and 7 of front 3 contain contaminant carrier 11 and 13 for attracting and holding airborne contaminants. Back 15 includes an adhesive for affixing device 1 to a person.

FIG. 2 shows a front perspective view of another present invention device 20, with front 21 having an identifier bar code 25, and contaminant carrier 27 covering the entire surface, including the bar code. Back 23 has an adhesive thereon for attachment to a user.

FIG. 3 illustrates a front perspective view of another present invention device 30. Here, the device has a round shape to illustrate that any shape may be used. The device 30 has a flexible plastic substrate, e.g. such as a plastic bandage (Band Aid®) by Johnson & Johnson. The front is divided into upper half 33, with an airborne contaminant carrier 34, and lower half 31, with a bar code identifier 35. Back 37 includes an adhesive for attachment to skin, clothing or a user's equipment.

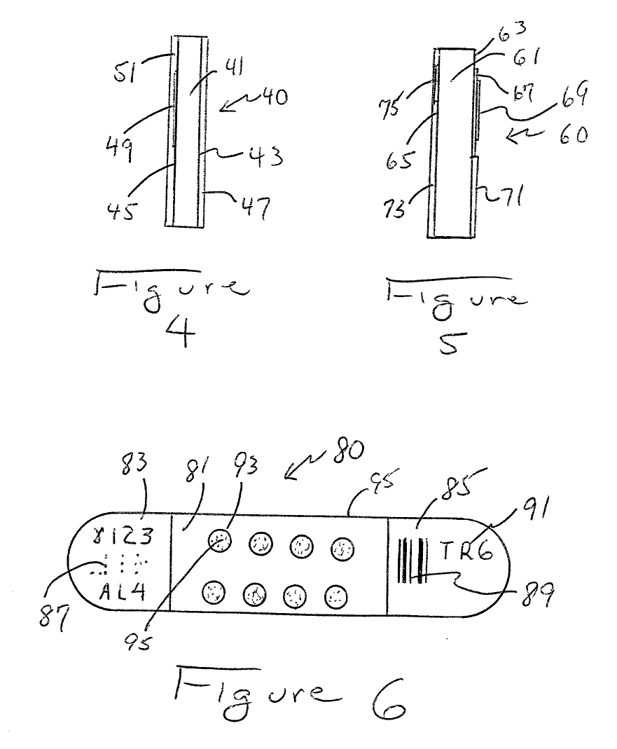

FIGS. 4 and 5 show side views of alternative embodiment present invention devices. FIG. 4 shows device 40, with flexible substrate 41. On front 43 is an airborne contaminant carrier 47 and on back 45 is an identifier 49 and an adhesive 51. This illustrates a hidden identifier on the back 45.

FIG. 5 illustrates device 60 with a flexible substrate 61 having a front 63 and a back 65. Front 63 includes an airborne contaminant carrier 71 and an identifier adhered thereto. Thus, identifier 69 was separately printed on its adhesive-backed substrate 67, which is attached to front 63. Back 65 has adhesive 73 for attachment to a user, and another identifier 75. This enables mass promoting of devices with the backside identifier, e.g.a specific army unit, a company or plant location, a mine, etc. and the front add-on identifier may be user and/or date specific.

FIG. 6 shows another present invention device 80 in a flexible bandage-type format and shape and could be packaged like a bandage. Front ends 83 and 85 have different identifier information 87, 89, and 91, as shown. Central front area 81 has a plurality of orifices with a backer strip and contains granular material, e.g. activated charcoal, or microporous polymer 95 exposed by orifices such as orifice 93. Back 95 has an adhesive for attachment.

A user will wear the present invention device and then take subsequent steps as directed. These steps may call for the user to actually submit the device to a portable or stationary analyzer; package and store it; package and submit it to a storage administrator; package and send it to a lab facility for testing, etc. The present invention devices may be stored in sealable, airtight plastic bags by the user to prevent undesired subsequent exposure of the device to which may be contaminated, and then handled has designated.

Obviously, numerous modifications and variations of the present invention are possible in light of the above teachings. It is therefore understood that within the scope of the appended claims, the invention may be practiced otherwise than as specifically described herein.

What is claimed is:

1. A device for use in detection of airborne contaminants, which comprises:
(a.) a flexible substrate having a front side and a back side;
(b.) a contaminant carrier located on said front side;
(c.) an adhesive located on said back side;
(d.) a unique identifier located on one of said front side and said back side.

2. The device of claim 1 wherein said contaminant carrier is a material adapted to receive and chemically bond with an airborne contaminant.

3. The device of claim 1 wherein said contaminant carrier is a material adapted to receive and ionically bond with an airborne contaminant.

4. The device of claim 1 wherein said contaminant carrier is a material adapted to receive and physically hold an airborne contaminant.

5. The device of claim 4 wherein said contaminant carrier is selected from the group consisting of adhesive material, high surface area material and combinations thereof.

6. The device of claim 5 wherein said high surface area material is selected from the group consisting of microporous polymeric material and powdered inorganic material.

7. The device of claim 1 wherein said unique identifier is selected form the group consisting of representations of alphabet, numerals, bar code, symbols, and combinations thereof.

8. The device of claim 1 wherein said unique identifier is located on said front side.

9. The device of claim 1 wherein said unique identifier is located on said back side.

10. The device of claim 1 wherein said unique identifier is located in a first area of said front side, and said carrier is located in a second area of said front side.

11. A method of creating and storing airborne contaminant samples and identification data for subsequent detection, which comprises:
(A.) applying a device for use in detection of airborne contaminants to a person, wherein said device includes:
(a.) a flexible substrate having a front side and a back side;
(b.) a contaminant carrier located on said front side;
(c.) an adhesive located on said back side;
(d.) a unique identifier located on one of said front side and said back side;
(B.) subjecting said person with said device, to an airborne contaminant wherein said airborne contaminant is received and held by said device; and,
(C.) subsequently subjecting said device with said airborne contaminant, to a detection method, and correlating results from said detection method to said unique identifier.

12. The method of claim 11 wherein said applying of said device to said person by attaching said device to at least one element selected from the group consisting of skin of said person, clothing of said person, equipment of said person and a secondary holder attachable to one of the foregoing.

13. The method of claim 12 wherein said device is attached to skin of said person.

14. The method of claim 11 wherein said contaminant carrier is a material adapted to receive and chemically bond with an airborne contaminant.

15. The method of claim 11 wherein said contaminant carrier is a material adapted to receive and ionically bond with an airborne contaminant.

16. The method of claim 11 wherein said contaminant carrier is a material adapted to receive and physically hold an airborne contaminant.

17. The method of claim 11 wherein said contaminant carrier is selected from the group consisting of adhesive material, high surface area material and combinations thereof.

18. The method of claim 11 wherein said high surface area material is selected form the group consisting of microporous polymeric material and powdered inorganic material.

19. The method of claim 11 wherein said unique identifier is selected form the group consisting of representations of alphabet, numerals, bar code, symbols, and combinations thereof.

20. The method of claim 11 wherein said unique identifier is located in a first area of said front side and said carrier is located in a second area of said front side.
